(19) United States
(12) Patent Application Publication
(10) Pub. No.: US 2024/0350068 A1
(43) Pub. Date: Oct. 24, 2024

(54) MEASURING QUALITY EVALUATING DEVICE, METHOD, AND PROGRAM

(71) Applicant: Nippon Telegraph and Telephone Corporation, Tokyo (JP)

(72) Inventors: Takayuki Ogasawara, Tokyo (JP); Kentaro Tanaka, Tokyo (JP); Nobuaki Matsuura, Tokyo (JP); Toichiro Goto, Tokyo (JP); Masumi Yamaguchi, Tokyo (JP)

(21) Appl. No.: 18/686,117

(22) PCT Filed: Oct. 5, 2021
(86) PCT No.: PCT/JP2021/036762

§ 371 Date: Feb. 23, 2024

(51) Int. Cl. A61B 5/352; A61B 5/308; A61B 5/339

(52) U.S. Cl. CPC A61B 5/352; A61B 5/308; A61B 5/339

(57) ABSTRACT

A first arithmetic circuit detects R waves from an electrocardiogram waveform measured by electrocardiograph and obtains an interval (heartbeat interval: RRI) between adjacent R waves. A second arithmetic circuit obtains an instantaneous heart rate for each beat indicated by an R wave from the interval obtained by the first arithmetic circuit. A third arithmetic circuit obtains a smoothed heart rate obtained by smoothing the instantaneous heart rate for each beat calculated by the second arithmetic circuit. A fourth arithmetic circuit obtains a signal-to-noise ratio of the electrocardiogram waveform using the instantaneous heart rate and the smoothed heart rate.

### CROSS-REFERENCE TO RELATED APPLICATIONS

This application is a national phase entry of PCT Application No. PCT/JP2021/036762, filed on Oct. 5, 2021, which application is hereby incorporated herein by reference.

### TECHNICAL FIELD

The present invention relates to a measurement quality evaluation device, method, and program for obtaining a signal-to-noise ratio in an electrocardiogram.

### BACKGROUND

In recent years, a technique has been proposed for detecting electrocardiogram information of a subject from a sensor worn by the user (subject) (NPL 1). In such a technique, electrocardiogram information is generally mixed with artifacts caused by the user's body motion, which may reduce the measurement quality, i.e. the clarity of the electrocardiogram shape. As a method of evaluating the measurement quality based on the validity of the heart rate value calculated from the electrocardiogram, for example, the technique of PTL 1 has been proposed to deal with the deterioration of the measurement quality.

### CITATION LIST

### Patent Literature

PTL 1-Japanese Patent Application Publication No. 2018-11819

### Non Patent Literature

NPL 1-NIPPON TELEGRAPH AND TELEPHONE CORPORATION, “Development of a low-power, compact wearable sensor that enables measurement of electrocardiogram, acceleration, temperature and humidity for smart healthcare,” NTT Holdings News Release, Nov. 8, 2019, [retrieved on Sep. 2, 2021], (https://www.ntt.co.jp/news2019/1911/191108a.html).

### SUMMARY

### Technical Problem

When evaluating the measurement quality in this way, the technique of PTL 1 evaluates the validity of the heart rate value, so the quality of the underlying electrocardiogram remains unknown. Therefore, information such as the magnitude of artifacts mixed in the electrocardiogram, which may have contributed to the deterioration of the quality of the electrocardiogram, is not available, and the user is unable to take corrective actions that lead to fundamental countermeasures.

Embodiments of the present invention have been made to solve the above problems, and an object of embodiments of the present invention is to provide a high-quality electrocardiogram.

### Solution to Problem

A measurement quality evaluation device according to embodiments of the present invention includes: a first arithmetic circuit that detects R waves from an electrocardiogram waveform measured by electrocardiograph and obtains an interval between adjacent R waves; a second arithmetic circuit that obtains an instantaneous heart rate for each beat indicated by an R wave from the interval obtained by the first arithmetic circuit; a third arithmetic circuit that obtains a smoothed heart rate obtained by smoothing the instantaneous heart rate for each beat calculated by the second arithmetic circuit; a fourth arithmetic circuit that obtains a signal-to-noise ratio of the electrocardiogram waveform using at least one of the instantaneous heart rate and the smoothed heart rate; and a transmission circuit that transmits the signal-to-noise ratio obtained by the fourth arithmetic circuit to a set destination.

A measurement quality evaluation method according to embodiments of the present invention includes: a first step of detecting R waves from an electrocardiogram waveform measured by electrocardiograph and obtaining an interval between adjacent R waves; a second step of obtaining an instantaneous heart rate for each beat indicated by an R wave from the interval obtained in the first step; a third step of obtaining a smoothed heart rate obtained by smoothing the instantaneous heart rate for each beat calculated in the second step; a fourth step of obtaining a signal-to-noise ratio of the electrocardiogram waveform using the instantaneous heart rate and the smoothed heart rate; and a fifth step of transmitting the signal-to-noise ratio obtained in the fourth step to a set destination.

A program according to embodiments of the present invention is a program for a computer to execute the measurement quality evaluation method described above.

### Advantageous Effects of embodiments of Invention

As described above, according to embodiments of the present invention, since the signal-to-noise ratio of the electrocardiogram waveform is obtained using the instantaneous heart rate and the smoothed heart rate, a high-quality electrocardiogram can be provided.

### BRIEF DESCRIPTION OF THE DRAWINGS

FIG. 1 is a configuration diagram showing the configuration of a measurement quality evaluation device according to Embodiment 1 of the present invention.

FIG. 2 is a flowchart illustrating a measurement quality evaluation method according to Embodiment 1 of the present invention.

FIG. 3A is a correlation diagram illustrating the correlation between the SNR obtained (estimated) from the training results of machine learning using the instantaneous heart rate, the smoothed heart rate and the SNR, and the SNR in training data for the SNR used for machine learning.

FIG. 3B is a correlation diagram showing the correlation between the SNR obtained (estimated) from the training results of machine learning using the instantaneous heart rate, the smoothed heart rate and the SNR, and the SNR in training data for the SNR used for machine learning.

FIG. 4 is a configuration diagram illustrating a hardware configuration of the measurement quality evaluation device.

FIG. 5 is a configuration diagram illustrating a system using the measurement quality evaluation device according to Embodiment 1.

FIG. 6 is a diagram illustrating a configuration of a system using the measurement quality evaluation device according to Embodiment 1.

FIG. 7 is a configuration diagram illustrating a configuration of a measurement quality evaluation device according to Embodiment 2 of the present invention.

FIG. 8 is a characteristic diagram illustrating one example of determination information used in the measurement quality evaluation method according to Embodiment 2.

FIG. 9 is a configuration diagram illustrating a configuration of a measurement quality evaluation device according to Embodiment 3 of the present invention.

FIG. 10 is a configuration diagram illustrating a configuration of a measurement quality evaluation device according to Embodiment 5 of the present invention.

FIG. 11A is a correlation diagram illustrating a relationship between SNR and RRI when SNR>24.

FIG. 11B is a correlation diagram illustrating a relationship between SNR and RRI when SNR=12.

FIG. 11C is a correlation diagram illustrating a relationship between SNR and RRI when SNR=−6.

### DETAILED DESCRIPTION OF ILLUSTRATIVE EMBODIMENTS

A measurement quality evaluation device according to an embodiment of the present invention will be described below.

### Embodiment 1

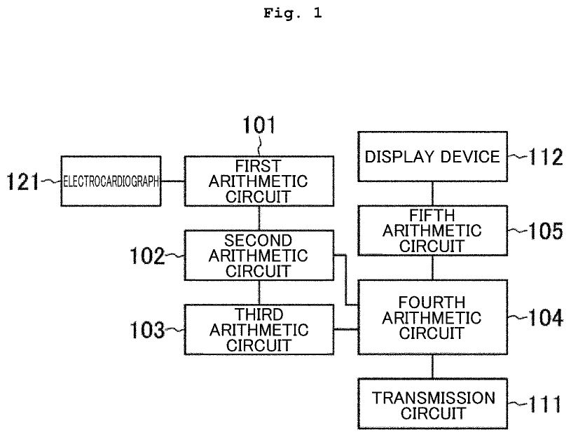

First, a measurement quality evaluation device according to Embodiment 1 of the present invention will be described with reference to FIG. 1 This measurement quality evaluation device includes a first arithmetic circuit 101, a second arithmetic circuit 102, a third arithmetic circuit 103, a fourth arithmetic circuit 104, and a transmission circuit 111.

The first arithmetic circuit 101 detects R waves from an electrocardiogram waveform (electrocardiogram) measured by electrocardiograph 121 and obtains an interval (heartbeat interval: RRI) between adjacent R waves. The second arithmetic circuit 102 obtains an instantaneous heart rate for each beat indicated by an R wave from the interval obtained by the first arithmetic circuit 101. The third arithmetic circuit 103 obtains a smoothed heart rate obtained by smoothing the instantaneous heart rate for each beat calculated by the second arithmetic circuit 102.

The fourth arithmetic circuit 104 obtains a signal-to-noise ratio of the electrocardiogram waveform using at least one of the instantaneous heart rate and the smoothed heart rate. The transmission circuit 111 transmits the signal-to-noise ratio obtained by the fourth arithmetic circuit 104 to a set destination.

The measurement quality evaluation device can include a fifth arithmetic circuit 105. A fifth arithmetic circuit 105 obtains a period during which the signal-to-noise ratio obtained by the fourth arithmetic circuit 104 is below a certain value within a measurement period during which the electrocardiogram information is measured, and displays the period on a display device 112. The fifth arithmetic circuit 105 obtains a ratio between the measurement period during which the electrocardiogram information is measured and a period during which the signal-to-noise ratio obtained by the fourth arithmetic circuit 104 is below a certain value in the measurement period, and displays the ratio on a display device 112.

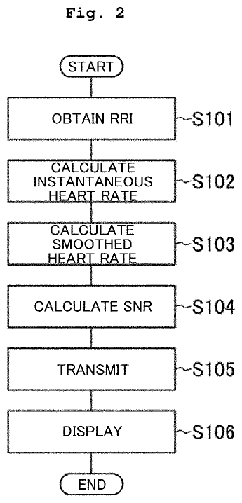

A measurement quality evaluation method (operation of the measurement quality evaluation device) according to Embodiment 1 of the present invention will be described with reference to FIG. 2

In a first step S101, the first arithmetic circuit 101 detects R waves from an electrocardiogram waveform measured by the electrocardiograph 121, and calculates intervals between adjacent R waves. For example, the first arithmetic circuit 101 can calculate the RRI from the electrocardiogram by using the peak detection of Reference 1.

In the second step S102, the second arithmetic circuit 102 calculates the instantaneous heart rate for each beat indicated by the R wave from the interval obtained in the first step S101. For example, as a method of calculating the instantaneous heart rate from the RRI, the instantaneous heart rate can be obtained by dividing 60000 by the RRI. Since the unit of instantaneous heart rate is beats per minute (bpm), it is established that: instantaneous heart rate [bpm(beats/minute)]=60[seconds/minute]×1000[milliseconds/second]/RRI [milliseconds/beat].

In a third step S103, the third arithmetic circuit 103 calculates a smoothed heart rate by smoothing the instantaneous heart rate for each beat obtained in the second step S102. For example, the smoothed heart rate can be a moving average using the most recent instantaneous heart rate data. A value obtained by applying an FIR filter or an IIR filter to the most recent instantaneous heart rate can be used as the smoothed heart rate.

In a fourth step S104, the fourth arithmetic circuit 104 calculates a signal-to-noise ratio of the electrocardiogram waveform using the instantaneous heart rate and the smoothed heart rate.

Machine learning, for example, can be used as a method of calculating the signal-to-noise ratio (SNR). Electrocardiogram data with few artifacts and noise data in which only artifacts are recorded are prepared in advance, and training data is created by synthesizing these pieces of data.

By synthesizing noise data with electrocardiogram data at different amplitude values, data sets of various SNRs are created. For electrocardiogram data, publicly available data disclosed in Reference 2, for example, may be used. For noise data, publicly available data disclosed in Reference 3, for example, may be used. In Reference 3, as an example of creating data sets with various SNRs, six types of data sets are provided by synthesizing the electrocardiogram data and noise data in reference 2 and changing the SNR from −6 to 24 dB. Therefore, this may be used as the SNR data set without modification.

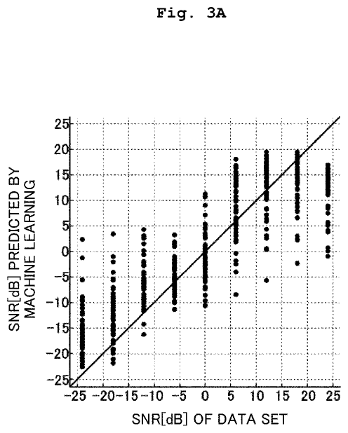

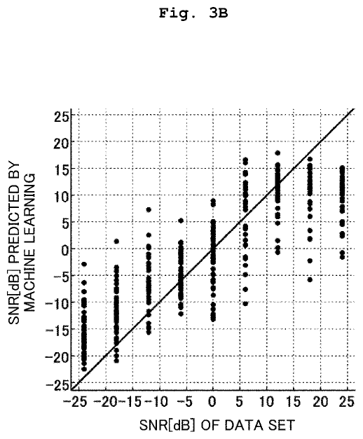

FIG. 3A is a correlation diagram illustrating the correlation between the SNR obtained (estimated) from the training results of machine learning using the instantaneous heart rate, the smoothed heart rate and the SNR, and the SNR in training data for the SNR used for machine learning. There is a positive correlation between the SNR of the data set and the predicted SNR, the coefficient of determination is 0.75, and the error (RMSE) is 7.83, which makes it possible to predict the SNR very well. FIG. 3A shows the correlation trained using the electrocardiogram data of Reference 2 and the noise data of Reference 4 with random forest, which is machine learning.

In this way, the SNR of the electrocardiogram can be calculated by using the determination information obtained by performing machine learning in advance using the instantaneous heart rate, the smoothed heart rate, and various SNR data sets.

Machine learning can also use only instantaneous heart rate and SNR data sets without using smoothed heart rate. FIG. 3B shows the learning result in this case. FIG. 3B shows the correlation trained using the electrocardiogram data of Reference 2 and the noise data of Reference 4 with random forest, which is machine learning. The coefficient of determination is 0.79, which is good as before, but the error (RMSE) is 8.00, which is slightly deteriorated because the smoothed heart rate is not used. However, it has the advantage of being easier to implement because it reduces the number of variables used for input by one. Further, machine learning is not limited to random forests, and support vector machines, neural networks, logistic regression or ensemble learning can be used. The signal-to-noise ratio of the electrocardiogram waveform can be obtained by multiple regression analysis.

In a fifth step S105, the transmission circuit 111 transmits the signal-to-noise ratio obtained in the fourth step S104 to a set destination. In a sixth step S106, the fifth arithmetic circuit 105 obtains a period during which the signal-to-noise ratio obtained is below a certain value within a measurement period during which the electrocardiogram information is measured, and displays the period on a display device 112. The fifth arithmetic circuit 105 obtains a ratio between the measurement period during which the electrocardiographic information is measured and a period during which the signal-to-noise ratio obtained is below a certain value in the measurement period, and displays the ratio on a display device 112.

Thus, the measured quality of the electrocardiogram can be appropriately provided to the user by displaying and outputting the calculated SNR, such as the period during which the SNR fell below a certain value within the measurement period in which electrocardiogram information was measured, or the ratio between the measurement period in which electrocardiogram information was measured and the period during which the SNR fell below a certain value.

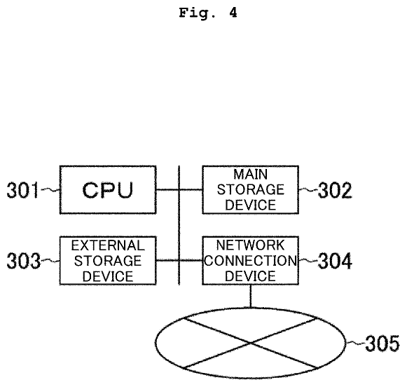

As shown in FIG. 4, the measurement quality evaluation device described above is a computer device equipped with a CPU (Central Processing Unit) 301, a main storage device 302, an external storage device 303, a network connection device 304 and the like. The functions described above (measurement quality evaluation method) can be implemented by the CPU 301 operating (executing the program) according to the program deployed in the main storage device 302. The program is a program for a computer to execute the measurement quality evaluation method shown in the above embodiment. The network connection device 304 is connected to the network 305. Also, each function can be distributed among a plurality of computer equipment.

Moreover, the measurement quality evaluation device according to the above-described embodiments can also be configured by PLD (programmable logic device) such as FPGA (field-programmable gate array). For example, by providing the logic element of the FPGA with a storage unit, a first arithmetic circuit, a second arithmetic circuit, a third arithmetic circuit, a fourth arithmetic circuit, and a transmission circuit, it can function as a device. Each of the memory circuit, the first arithmetic circuit, the second arithmetic circuit, the third arithmetic circuit, the fourth arithmetic circuit, and the transmission circuit can be written to the FPGA by connecting a predetermined writing device. Moreover, each of the above circuits written in the FPGA can be confirmed by a writing device connected to the FPGA.

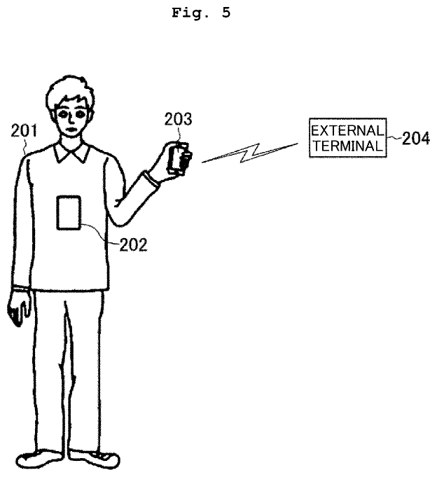

A system using the measurement quality evaluation device according to Embodiment 1 will be described. For example, as shown in FIG. 5, a sensor terminal 202 is attached to the trunk of a subject 201, and the results measured by the electrocardiograph built in the sensor terminal 202 are transmitted to an external terminal 204 via a relay terminal 203. The sensor terminal 202 can be a computer device such as a smart phone and a tablet. The relay terminal 203 and the external terminal 204 can be computer device such as a server device.

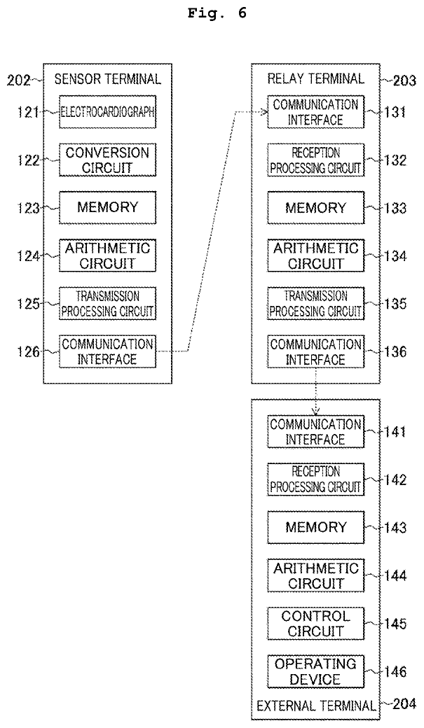

The sensor terminal 202 includes an electrocardiograph 121, a conversion circuit 122, a memory 123, an arithmetic circuit 124, a transmission processing circuit 125, and a communication interface 126, as shown in FIG. 6. The relay terminal 203 includes a communication interface 131, a reception processing circuit 132, a memory 133, an arithmetic circuit 134, a transmission processing circuit 135 and a communication interface 136 The external terminal 204 includes a communication interface 141, a reception processing circuit 142, a memory 143, an arithmetic circuit 144, a control circuit 145 and an operating device 146.

The conversion circuit 122 converts the analog acceleration signal measured by the electrocardiograph 121 into digital data at a predetermined sampling rate and outputs the data. The memory 123 stores the electrocardiogram waveform digitized by the conversion circuit 122. The arithmetic circuit 124 obtains the RRI based on the electrocardiogram waveform stored in the memory 123. The arithmetic circuit 124 obtains an instantaneous heart rate for each beat indicated by the R wave from the obtained RRI. The arithmetic circuit 124 obtains a smoothed heart rate obtained by smoothing the obtained instantaneous heart rate for each beat. The arithmetic circuit 124 obtains a signal-to-noise ratio of the electrocardiogram waveform using the instantaneous heart rate and the smoothed heart rate. For example, the arithmetic circuit 124 can include a first arithmetic circuit 101, a second arithmetic circuit 102, a third arithmetic circuit 103, and a fourth arithmetic circuit 104.

The transmission processing circuit 125 transmits the processing result (for example, RRI) processed by the arithmetic circuit 124 to the relay terminal 203 via the communication interface 126. The communication interface 126 is composed of an arithmetic interface and an antenna compatible with wireless data communication protocols such as LTE (Long Term Evolution), third-generation mobile communication systems, wireless LAN (Local Area Network), and Bluetooth (registered trademark).

The relay terminal 203 consists of a communication interface 131 that receives data transmitted from the sensor terminal 202, a reception processing circuit 132, a memory 133, an arithmetic circuit 134, a transmission processing circuit 135, and a communication interface 136 that transmits data to the external terminal 204.

The external terminal 204 has a communication interface 141 that receives data transmitted from the relay terminal 203, a reception processing circuit 142, a memory 143, an arithmetic circuit 144, and a control circuit 145 that instructs the operating device 146, which operates based on the analyzed data, to give operating instructions.

The control circuit 145 causes the operating device 146 to perform actions to assist the subject based on the information stored in the memory 143 (electrocardiogram waveform and signal-to-noise ratio of the electrocardiogram waveform).

The operating device 146 includes an image output device (e.g. monitor), an audio output device (e.g. speaker or musical instrument), a light source (e.g. light emitting diodes (LEDs) or electric bulbs), an actuator (vibrator, robot arm or electric therapy device), and a thermal device (heater or Peltier element).

It is not necessary for the arithmetic circuit 124 to include all of the first arithmetic circuit 101, the second arithmetic circuit 102, the third arithmetic circuit 103 and the fourth arithmetic circuit 104, which can be distributed to the arithmetic circuits 134 and 144.

### Embodiment 2

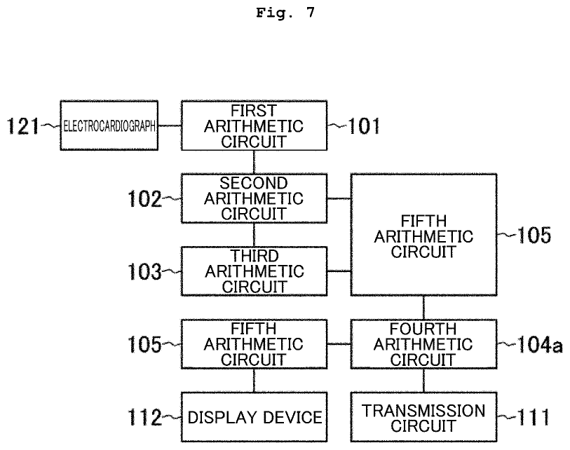

A measurement quality evaluation device according to Embodiment 2 of the present invention will be described with reference to FIG. 7. This measurement quality evaluation device includes a first arithmetic circuit 101, a second arithmetic circuit 102, a third arithmetic circuit 103, a fourth arithmetic circuit 104a, a transmission circuit 111, a fifth arithmetic circuit 105, and a sixth arithmetic circuit 106. The first arithmetic circuit 101, the second arithmetic circuit 102, the third arithmetic circuit 103, the fifth arithmetic circuit 105, and the transmission circuit 111 are the same as those in Embodiment 1 described above.

The sixth arithmetic circuit 106 determines adequacy of the smoothed heart rate obtained by the third arithmetic circuit 103. The fourth arithmetic circuit 104a obtains the signal-to-noise ratio of the electrocardiogram waveform based on the determination result of the sixth arithmetic circuit 106.

In a measurement quality evaluation method according to Embodiment 2, in a first step, first arithmetic circuit 101 detects R waves from an electrocardiogram waveform measured by electrocardiograph 121, and calculates the interval between adjacent R waves. In a second step, the second arithmetic circuit 102 calculates the instantaneous heart rate for each beat indicated by the R wave from the interval obtained in the first step.

In a third step, the third arithmetic circuit 103 calculates a smoothed heart rate by smoothing the instantaneous heart rate for each beat obtained in the second step S102. In a sixth step, the sixth arithmetic circuit 106 determines adequacy of the smoothed heart rate obtained in the third step. In a fourth step, the fourth arithmetic circuit 104a obtains the signal-to-noise ratio of the electrocardiogram waveform based on the determination result in the sixth step.

Determining the adequacy of the smoothed heart rate can be implemented by using the method described in claim 1 of PTL 1, for example. The output from the sixth arithmetic circuit 106 (i.e. the determination result of either being adequate or inadequate) and the determination information for calculating SNR using the aforementioned SNR data set should be created in advance by machine learning, which should be stored by the fourth arithmetic circuit 104a.

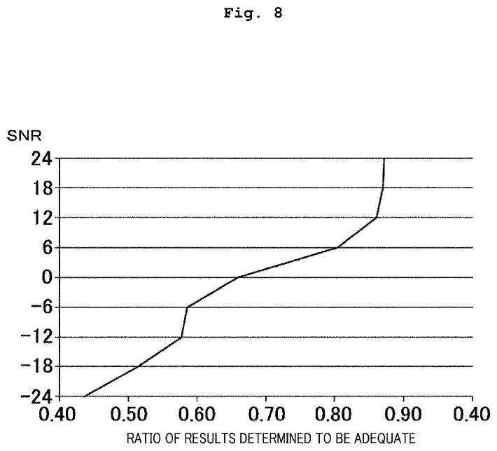

FIG. 8 illustrates one example of the determination information. In FIG. 8, the vertical axis is the SNR, and the horizontal axis is the ratio of results determined to be adequate in the appropriateness determination results. For example, if 60 determination results are obtained per minute and 50 of them are determined to be appropriate, the value corresponding to the horizontal axis is 50/60≈0.83. In this case, it is found that the SNR is about 8 dB according to the relationship shown in FIG. 8. If the value corresponding to the horizontal axis is less than 0.44 or greater than 0.87, the SNR cannot be obtained from FIG. 8. However, in these cases, an output that can be regarded as less than −24 dB and greater than 24 dB should be given.

By obtaining the signal-to-noise ratio of the electrocardiogram waveform based on the decision information learned in advance by machine learning based on the adequacy of the instantaneous heart rate, existing technologies such as those described in PTL 1 can be effectively utilized to provide users with an implementation option that provides appropriate electrocardiogram measurement quality. In addition, the configuration of the fourth arithmetic circuit 104a can be simplified because only one input is required in Embodiment 2, whereas two inputs were required for the fourth arithmetic circuit 104 in Embodiment 1.

### Embodiment 3

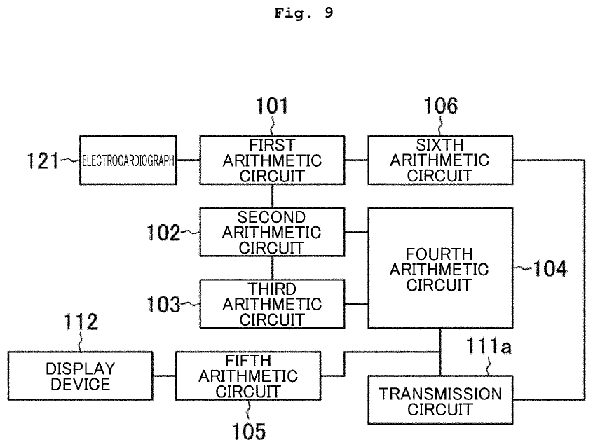

A measurement quality evaluation device according to Embodiment 3 of the present invention will be described with reference to FIG. 9. This measurement quality evaluation device includes a first arithmetic circuit 101, a second arithmetic circuit 102, a third arithmetic circuit 103, a fourth arithmetic circuit 104, a fifth arithmetic circuit 105, a transmission circuit 111a, and a seventh arithmetic circuit 107. The first arithmetic circuit 101, the second arithmetic circuit 102, the third arithmetic circuit 103, the fourth arithmetic circuit 104, and the fifth arithmetic circuit 105 are the same as those in Embodiment 1 described above.

A seventh arithmetic circuit 107 generates non-computable information when the first arithmetic circuit 101 does not detect the R wave for the set period of time, The transmission circuit 111a transmits the non-computable information.

In a measurement quality evaluation method according to Embodiment 3, in a first step, first arithmetic circuit 101 detects R waves from an electrocardiogram waveform measured by electrocardiograph 121, and calculates the interval between adjacent R waves. In a second step, the second arithmetic circuit 102 calculates the instantaneous heart rate for each beat indicated by the R wave from the interval obtained in the first step.

In a third step, the third arithmetic circuit 103 calculates a smoothed heart rate by smoothing the instantaneous heart rate for each beat obtained in the second step S102. In a seventh step, the seventh arithmetic circuit 107 generates non-computable information when the R wave is not detected for the set period of time in the first step. In a fourth step, the fourth arithmetic circuit 104 calculates a signal-to-noise ratio of the electrocardiogram waveform using the instantaneous heart rate and the smoothed heart rate. In the fifth step, the transmission circuit 111 transmits the signal-to-noise ratio obtained in the fourth step and the non-computable information generated in the seventh step to the set destination.

In Embodiment 1 described above, the SNR is calculated on the assumption that the RRI can be obtained as a value. Therefore, if the R wave cannot be detected and the RRI cannot be obtained, the SNR cannot be calculated. In order to deal with such cases, the seventh arithmetic circuit 107 generates non-computable information indicating that the RRI has not been detected when the RRI is not obtained within a certain period of time (for example, two seconds exceeding the beat cycle of a typical heartbeat). The non-computable information will be signaled as an alternative for which the SNR is not updated. As a result, it is possible to notify that there is a problem in the measurement, that is, that the measurement quality is not good even in a situation where the SNR cannot be obtained.

### Embodiment 4

A measurement quality evaluation device according to Embodiment 4 of the present invention will be described below. This measurement quality evaluation device is a modification of Embodiments 1 to 3 described above. Embodiment 4 is characterized in that when data from a sensor terminal is lost due to communication interruption (packet loss) in a reception processing circuit in a relay terminal or an external terminal, loss information is notified to a memory.

If the SNR obtained in the aforementioned forms or the non-computable information determined in Embodiment 3 is lost as packet loss when transferred to a relay terminal or an external terminal, the information will not reach the user. However, if the occurrence of the packet loss is stored and notified, it is not necessary to determine that there is a problem in the data detection and analysis process of the sensor terminal. Thus, accurate information on the cause can be provided even in a situation where the information on the SNR does not reach the user.

### Embodiment 5

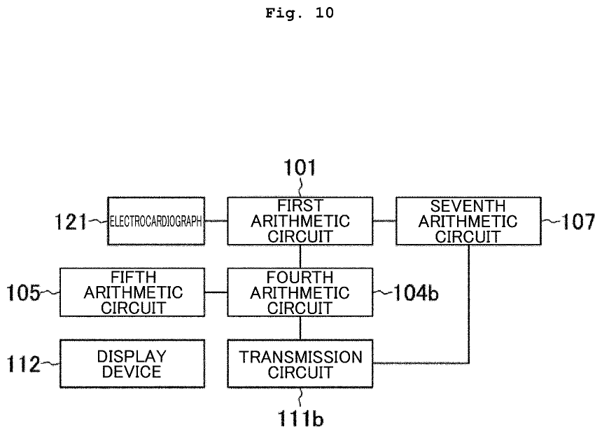

A measurement quality evaluation device according to Embodiment 5 of the present invention will be described with reference to FIG. 10. This measurement quality evaluation device includes a first arithmetic circuit 101, a fourth arithmetic circuit 104b, a fifth arithmetic circuit 105, a transmission circuit 111b and an eighth arithmetic circuit 108. The first arithmetic circuit 101 and the fifth arithmetic circuit 105 are the same as in Embodiment 1 described above.

The eighth arithmetic circuit 108 calculates SNR based on the RRI calculated by the first arithmetic circuit 101, or determines arrhythmia based on the RRI. The transmission circuit 111b transmits the SNR calculated by the eighth arithmetic circuit 108.

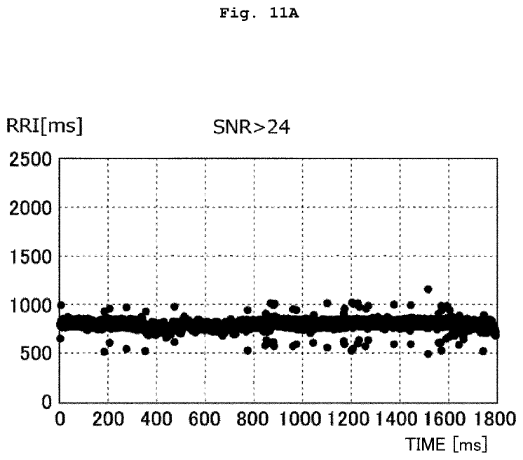

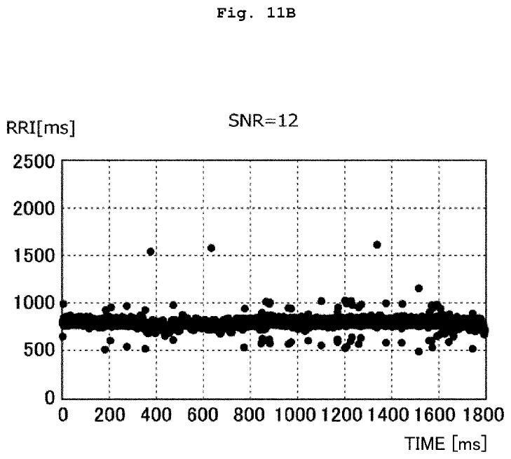

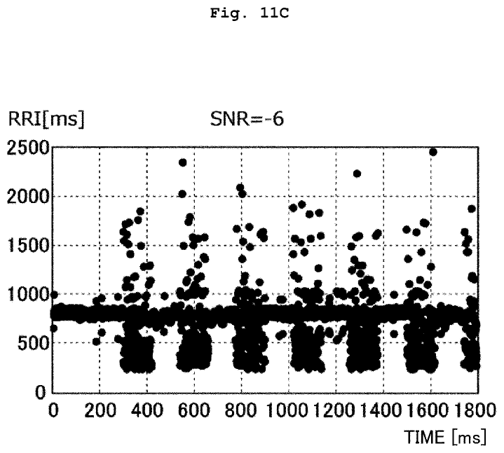

FIGS. 11A, 11B, and 11C show the relationship between SNR and RRI. In the graph of SNR>24 in FIG. 11A, the RRI changes around 800 ms, but there are cases where it shows around 1000 ms or 500 ms, which is due to fluctuations due to arrhythmia.

However, as SNR=12 in FIG. 11B and SNR=−6 in FIG. 11C, the number of points greatly deviating from RRI 800 ms increases. This is because the first arithmetic circuit 101 erroneously detected the artifact as the R wave.

Focusing on this tendency, the SNR can be grasped by using a threshold. For example, the degree of SNR can be estimated from RRI as “high SNR” when RRI is in the range of 400<RRI<1200, “medium SNR” when 1200<RRI<1700, and “low SNR” when RRI<400 or 1700<RRI. It is also possible to estimate from the RRI value whether the variation is due to arrhythmia, for example, if the RRI deviates from the mean value by 100 to 200 ms, it is possible to discriminate between arrhythmia and artifact, such as “likely arrhythmia” if the RRI is in the range 400<RRI<1200 or “likely artifact” if the RRI is RRI<400 or 1200<RRI.

When the measured electrocardiogram waveform (electrocardiogram) is recorded over multiple days, the average processing circuit is provided to calculate the average time sequence using the RRI values calculated from the electrocardiogram at the same time period on each day. The average processing circuit calculates the average time sequence using only the instantaneous heart rate or the smooth heart rate when the SNR calculated in the fourth calculation circuit is above a certain value.

For the average processing circuit described above, the technique of Reference 4, for example, may be used. This makes it possible to handle only the highly reliable instantaneous heart rate or smoothed heart rate detected under high SNR conditions, and to calculate a highly reliable average time series.

As described above, according to embodiments of the present invention, since the signal-to-noise ratio of the electrocardiogram waveform is obtained using the instantaneous heart rate and the smoothed heart rate, a high-quality electrocardiogram can be provided. According to embodiments of the present invention, it is possible to estimate the magnitude of artifacts mixed in an electrocardiogram waveform, and to provide a high-quality electrocardiogram waveform.

Note that it is clear that the present invention is not limited to the embodiments described above and within the technical concept of the present invention and many modifications and combinations can be implemented by those skilled in the art.
Reference 1—Analog Devices, “Let's create healthcare IoT that visualizes stress using electrocardiographic sensors!”, IoT course for web engineers taught by hardware professionals, 8th, [retrieved on Sep. 2, 2021], (https://www.analog.com/jp/education/landing-pages/003/iot_lectureship/content_08.html).
Reference 2—G. Moody and R. Mark, “MIT-BIH Arrhythmia Database”, [retrieved on Sep. 2, 2021], (https://physionet.org/content/mitdb/1.0.0/).
Reference 3—G. Moody and R. Mark, “MIT-BIH Noise Stress Test Database”, [retrieved on Sep. 2, 2021], (https://physionet.org/content/nstdb/1.0.0/).
Reference 4—Japanese Patent Application Publication No. 2020-036781

### REFERENCE SIGNS LIST

101: First arithmetic circuit
102: Second arithmetic circuit
103: Third arithmetic circuit
104: Fourth arithmetic circuit
105: Fifth arithmetic circuit
111: Transmission circuit
112: Display device
121: Electrocardiograph

1-8. (canceled)

9. A device, comprising:
a first arithmetic circuit configured to detect R waves from an electrocardiogram waveform measured by electrocardiograph and obtain an interval between adjacent R waves;
a second arithmetic circuit configured to obtain an instantaneous heart rate for each beat indicated by an R wave from the interval obtained by the first arithmetic circuit;
a third arithmetic circuit configured to obtain a smoothed heart rate by smoothing the instantaneous heart rate for each beat calculated by the second arithmetic circuit;
a fourth arithmetic circuit configured to obtain a signal-to-noise ratio of the electrocardiogram waveform based on the instantaneous heart rate or the smoothed heart rate; and
a transmission circuit configured to transmit the signal-to-noise ratio to a set destination.

10. The device according to claim 9, further comprising a fifth arithmetic circuit configured to:
obtain a period during which the signal-to-noise ratio is below a threshold value within a measurement period during which the electrocardiogram waveform is measured, and display the period on a display device.

11. The device according to claim 9 further comprising a fifth arithmetic circuit configured to:
obtain a ratio between a measurement period during which the electrocardiogram waveform is measured and a period during which the signal-to-noise ratio is below a threshold in the measurement period, and display the ratio on a display device.

12. The device according to claim 9, further comprising a sixth arithmetic circuit configured to determine adequacy of the smoothed heart rate obtained by the third arithmetic circuit, wherein the fourth arithmetic circuit obtains the signal-to-noise ratio of the electrocardiogram waveform based on a determination result of the sixth arithmetic circuit.

13. The device according to claim 9, further comprising a seventh arithmetic circuit configured to generate a non-computable indication in response to the first arithmetic circuit not detecting a R wave for a set period of time, wherein the transmission circuit is configured to transmit the non-computable indication.

14. A method, comprising:
a first step of detecting R waves from an electrocardiogram waveform measured by electrocardiograph and obtaining an interval between adjacent R waves;
a second step of obtaining an instantaneous heart rate for each beat indicated by an R wave from the interval obtained in the first step;
a third step of obtaining a smoothed heart rate by smoothing the instantaneous heart rate for each beat calculated in the second step;
a fourth step of obtaining a signal-to-noise ratio of the electrocardiogram waveform based on the instantaneous heart rate or the smoothed heart rate; and
a fifth step of transmitting the signal-to-noise ratio to a set destination.

15. The method according to claim 14, further comprising:
a sixth step of determining adequacy of the smoothed heart rate obtained in the third step, wherein the fourth step includes a step of obtaining the signal-to-noise ratio of the electrocardiogram waveform based on a determination result of the sixth step.

16. The method according to claim 14, further comprising:
a seventh step of generating a non-computable indication when an R wave is not detected for a set period of time in the first step; and
an eighth step of transmitting the non-computable indication.

17. The method according to claim 14 further comprising:
a ninth step of obtaining a period during which the signal-to-noise ratio is below a threshold value within a measurement period during which the electrocardiogram waveform is measured, and display the period on a display device.

18. The method according to claim 14 further comprising:
a tenth step of obtaining a ratio between a measurement period during which the electrocardiogram waveform is measured and a period during which the signal-to-noise ratio is below a threshold in the measurement period, and display the ratio on a display device.

19. A non-transitory computer-readable storage device storing a program that when executed by one or more processors, cause the one or more processors to execute:
a first step of detecting R waves from an electrocardiogram waveform measured by electrocardiograph and obtaining an interval between adjacent R waves;
a second step of obtaining an instantaneous heart rate for each beat indicated by an R wave from the interval obtained in the first step;
a third step of obtaining a smoothed heart rate by smoothing the instantaneous heart rate for each beat calculated in the second step;
a fourth step of obtaining a signal-to-noise ratio of the electrocardiogram waveform based on the instantaneous heart rate or the smoothed heart rate; and
a fifth step of transmitting the signal-to-noise ratio to a set destination.

20. The non-transitory computer-readable storage device according to claim 19, wherein the program when executed by the one or more processors, further cause the one or more processors to execute:
a sixth step of determining adequacy of the smoothed heart rate obtained in the third step, wherein the fourth step includes a step of obtaining the signal-to-noise ratio of the electrocardiogram waveform based on a determination result of the sixth step.

21. The non-transitory computer-readable storage device according to claim 19, wherein the program when executed by the one or more processors, further cause the one or more processors to execute:
a seventh step of generating a non-computable indication when an R wave is not detected for a set period of time in the first step; and
an eighth step of transmitting the non-computable indication.

22. The non-transitory computer-readable storage device according to claim 19, wherein the program when executed by the one or more processors, further cause the one or more processors to execute:
a ninth step of obtaining a period during which the signal-to-noise ratio is below a threshold value within a measurement period during which the electrocardiogram waveform is measured, and display the period on a display device.

23. The non-transitory computer-readable storage device according to claim 19, wherein the program when executed by the one or more processors, further cause the one or more processors to execute:
a tenth step of obtaining a ratio between a measurement period during which the electrocardiogram waveform is measured and a period during which the signal-to-noise ratio is below a threshold in the measurement period, and display the ratio on a display device.
